(19) United States
(12) Patent Application Publication
(10) Pub. No.: US 2010/0145026 A1
(43) Pub. Date: Jun. 10, 2010

(54) Antibody for ADCC And Inducing Cytokine Production

(75) Inventors: Christophe de ROMEUF, Lille (FR); Arnaud GLACET, Gondecourt (FR); Jacques LIROCHON, Breuillet (FR); Dominique BOUREL, La Madeleine (FR)

Correspondence Address: FOLEY AND LARDNER LLP;SUITE 500, 3000 K STREET NW, WASHINGTON, DC, 20007, US

(73) Assignee: LFB BIOTECHNOLOGIES

(21) Appl. No.: 12/576,202

(22) Filed: Oct. 8, 2009

Related U.S. Application Data
(63) Continuation of application No. 10/527,664, filed on Sep. 19, 2005, now abandoned.

(30) Foreign Application Priority Data
Sep. 13, 2002 (FR) 02/11415
Sep. 13, 2002 (FR) 02/11416
Jun. 12, 2003 (FR) 03/07067

(51) Int. Cl. C07K 16/00

(52) U.S. Cl. 530/387.3

(57) ABSTRACT

The invention concerns chimeric monoclonal antibodies, humanized or human produced in selected cell lines, said antibodies exhibiting high affinity for the CD16 receptor of effector cells of the immune system and hence capable of inducing high ADCC, but also the property of inducing cytokine and interleukin secretion, in particular IFNγ, which can enhance the ADCC activity of effector cells and hence be used for treating cancers and infections by pathogenic agents.

The present invention relates to human or humanized chimeric monoclonal antibodies which are produced in selected cell lines, said antibodies exhibiting high affinity for the CD16 receptor of effector cells of the immune system, but also the property of inducing the secretion of cytokines and of interleukins, which can modulate the cytotoxic activity of effector cells.

Immunotherapy by means of monoclonal antibodies is in the process of becoming one of the most important aspects of medicine. On the other hand, the results obtained in clinical trials appear to be contrasting. In fact, the monoclonal antibody may prove to be insufficiently effective. Many clinical trials are stopped for various reasons, such as a lack of effectiveness, and side effects that are incompatible with use in clinical therapy. These two aspects are closely linked, given that relatively inactive antibodies are administered at high doses so as to compensate and obtain a therapeutic response. The administration of a high dose induces not only side effects, but is also not very economically viable.

These are major problems in the industrial development of human or humanized chimeric monoclonal antibodies. By way of example, the company Protein Design Labs has suspended phase I/II clinical trials of Remitogen®, which is an anti-HLA-DR antibody that may be used for treating cancers of MHC class II-positive cells, in particular B-cell and T-cell leukemias.

Today, research is directed toward the immunoglobulin Fcγ fragment in order to improve the functional properties of the antibodies. In the end, this should make it possible to obtain antibodies which interact with and activate the receptors of effector cells (monocyte-macrophages, B lymphocytes, NK cells and dendritic cells).

The binding of an antibody to its ligand can induce activation of certain effector cells via their Fc receptors, which is the objective of the present invention. We have shown that certain antibodies not only have functional properties, such as ADCC or complement activation, but also induce the production of cytokines. These cytokines, produced at the site of effector activation, can exercise various biological activities.

Thus, it appears to be necessary to test the properties, of candidate antibodies, of inducing the production of these immune response-modulating factors. In fact, it has been found that the activation of effector cell receptors produces very different responses, resulting in the release of one or more cytokines. These cytokines are responsible for the activation or the inhibition of certain components of the immune system, as appropriate. It may be, for example, that the same antibody directed against a given antigen is completely ineffective when it is produced in mouse myeloma lines, whereas it is found to be very effective when it is produced in other cell lines.

For example, we demonstrate here that the binding of an antibody to its ligand can induce activation of CD16-transfected Jurkat cells, resulting in the secretion of IL2. A strong correlation is observed between the secretion of IL2 by Jurkat CD16 and the CD16-mediated ADCC activity of the effector cells.

We have shown, in our application WO 01/77181 (LFB), the importance of selecting cell lines for producing antibodies exhibiting strong ADCC activity of the FcγRIII (CD16) type. We have found that modifying the glycosylation of the constant fragment of the antibodies resulted in an improvement in the ADCC activity in rat myeloma lines such as YB2/0, the glycan structures being of the biantennary type, with short chains, a low degree of sialylation, nonintercalated terminal attachment point mannoses and GlcNAcs and a low degree of fucosylation.

Now, in the context of the present invention, we have discovered that the advantage of exhibiting a high affinity for CD16 can be further enhanced by additional tests aimed at choosing the antibodies that induce cytokine production.

The abovementioned two characteristics complement one another. In fact, the production of cytokines induced by the antibodies selected by means of the method of the invention could enhance the cytotoxic activity of antibodies. The mechanism of action of such an activation probably stems from a positive autocrine regulation of the effector cells. It may be postulated that the antibodies bind to CD16, bringing about a cytotoxic activity, but also induce the production of IFN gamma which, in the end, may result in an even further increase in the cytotoxic activity.

The invention therefore proposes antibodies which have an activity that is up to 100 times greater than the available antibodies in therapy. In particular, the invention provides an anti-HLD-DR and an anti-CD20 that are significantly more effective than their respective homologue such as Remitogen® and Rutixan®. The present invention marks a major turning point in the development of antibodies for clinical purposes, by providing a new generation, the effective doses of which in order to obtain 50% activity are much lower than those of the antibodies currently used.

### DESCRIPTION

Thus, the invention relates to a human or humanized chimeric monoclonal antibody produced in a cell line selected for its properties of glycosylation of the Fc fragment of an antibody, or the glycan structure of which has been modified ex vivo, said antibody having an FcγRIII (CD16)-type ADCC rate of greater than 60%, 70%, 80% or preferably greater than 90%, compared with the same antibody produced in a CHO line or with a commercially available homologous antibody, characterized in that it has an ability to induce a rate of production of at least one cytokine by a CD16 receptor-expressing effector cell of the immune system of greater than 60%, 100%, or preferably greater than 200%, compared with the same antibody produced in a CHO line or with a commercially available homologous antibody.

Preferably, this antibody has an ADCC rate of greater than 100% at a concentration of 10 ng/ml, compared with the same antibody produced in a CHO line or with a commercially available homologous antibody, and a rate of production of at least one cytokine by an effector cell of the immune system, in particular those expressing the CD16 receptor, of greater than 1000% at a concentration of 10 ng/ml, compared with the same antibody produced in a CHO line or with a commercially available homologous antibody.

Said cytokines that are released are interleukins, interferons and tissue necrosis factors (TNFs).

Thus, the antibody is selected for its ability to induce the secretion of at least one cytokine chosen from IL-1, IL-2, IL-3, IL-4, IL-5, IL-6, IL-7, IL-8, IL-9, IL-10, etc., TNFa, TGFβ, IP10 and IFNγ, by the effector cells of the immune system, in particular those expressing the CD16 receptor.

Preferably, the antibody selected has the ability to induce the secretion of IFNγ by the CD16 receptor-expressing effector cells of the immune system. The amount of IFNγ secreted reflects the quality of the antibody bound by the CD16 receptor as regards its antigen-binding integrity (Fc function) and capacity (antigenic site). In addition, the secretion of IFNγ contributes to enhancing the cytotoxic activity of the effector cells.

The effector cells may express an endogenous CD16 or may be transformed. The term “transformed cell” is intended to mean a cell that has been genetically modified so as to express Fc receptors, in particular the CD16 receptor.

In a particular embodiment, the antibody of the invention is capable of inducing the secretion of at least one cytokine by a leukocytic cell, in particular of the NK (natural killer) family, or by cells of the monocyte-macrophage group. Preferably, for selecting the antibodies, a Jurkat line transfected with an expression vector encoding the CD16 receptor is used as effector cell. This line is particularly advantageous since it is immortalized, i.e. it develops indefinitely in culture media, and it makes it possible to obtain reproducible results by virtue of its stability of CD16 expression.

In addition, the antibody can be selected after having been purified and/or modified ex vivo by modification of the glycan structure of the Fc fragment.

The selection can be carried out on antibodies produced by cells commonly used for the production of therapeutic antibodies, such as rat myeloma lines, in particular YB2/0 and its derivatives, human lymphoblastoid cells, insect cells and murine myeloma cells. The selection can also be applied to the evaluation of antibodies produced by transgenic plants or transgenic mammals. To this effect, production in CHO serves as a reference (CHO being used for the production of medicinal product antibodies) for comparing and selecting the production systems that result in the antibodies according to the invention. Another alternative consists in performing the comparison with commercially available antibodies, in particular antibodies in the process of being developed, antibodies that have obtained a marketing authorization or alternatively antibodies for which the clinical trials were stopped and which were found to be relatively ineffective and/or producing adverse side effects at the doses administered. In fact, as indicated above, the modified antibodies of the invention are up to 100 times more effective in activating the ADCC of effector cells of the immune system, which implies administration doses lower than those used with the antibodies mentioned above.

The antibody of the invention can be produced in cell lines of the rat myeloma type, in particular YB2/0. It can be directed against a normal, non-ubiquitous antigen (for example, the specificity of the antibody is anti-Rhesus D of the human red blood cell), or an antigen of a pathological cell or of an organism that is pathogenic for humans, in particular against an antigen of a cancer cell. By

In a preferred aspect, the antibody is an anti-HLA-DR. This antibody has an ADCC rate of greater than 100% at a concentration of 10 ng/ml or less, and a rate of IL-2 production by a CD16-receptor-expressing effector cell of the immune system of greater than up to 1000% at a concentration of 10 ng/ml, compared with the same antibody expressed in the CHO line, the expression line for Remitogen®.

The anti-HLA-DR of the invention can be produced in a rat myeloma line, in particular YB2/0.

In another preferred aspect, the antibody of the invention is an anti-CD20. This antibody has an ADCC rate of greater than 100% at a concentration of 10 ng/ml or less, and a rate of IL-2 production by Jurkat CD16 cells of greater than up to 1000% at a concentration of 10 ng/ml, compared with Rituxan®.

The anti-CD20 of the invention can be produced in a rat myeloma line, in particular YB2/0.

Other antibodies can be selected from anti-Ep-CAM, anti-KIR3DL2, anti-VEGFR, anti-HER1, anti-HER2, anti-GD, anti-GD2, anti-GD3, anti-CD23, anti-CD30, anti-CD33, anti-CD38, anti-CD44, anti-CD52, anti-CA125 and anti-ProteinC antibodies; anti-idiotypes specific for inhibitors, for example for clotting factors including FVIII and FIX, antivirals: HBV, HIV, HCV and RSV.

In another aspect, the invention relates to the use of an antibody described above, for producing a medicinal product intended for the treatment of cancers, for example anti-VEGFR, and of infections with pathogenic agents, for example anti-HIV.

More particularly, the invention is directed toward the use of an anti-HLA-DR or anti-CD20 antibody described above, for producing a medicinal product intended for the treatment of cancers of MHC class II-positive cells, in particular B-cell lymphomas, acute B-cell leukemias, Burkitt's lymphoma, Hodgkin's lymphoma, myeloid leukemias, T-cell lymphomas and leukemias, non-Hodgkin's lymphomas, and chronic myeloid leukemias.

In a preferred aspect of the invention, the antibody may, firstly, be selected for its CD16 receptor affinity, and then assayed and selected as described above for its properties of inducing the production of a cytokine, in particular IL-2, by Jurkat CD16 cells, or IFNγ by CD16-expressing effector cells from the blood.

Such antibodies having this double property of inducing ADCC via CD16 and of inducing the production of IL-2, result in a very substantial stimulation of the cytotoxic activity of effector cells. For example, this antibody may be an antibody listed above, produced in any cell line and selected by means of the assays mentioned above. These antibodies may be second-generation antibodies that are more effective than their currently available homologues (see table 1 below).

TABLE 1

| Antibody name | | | |
|---|---|---|---|
| and trade mark | Company | Target | Indication |
| Edrecolomab | Centocor | anti-Ep-CAM | colorectal |
| PANOREX | | | cancer |
| Rituximab | Idec | anti-CD20 | B cell lymphoma |
| RITUXAN | licensed to | | thrombocytopenia |
| | Genentech/ | | purpura |
| | Hoffmann la | | |
| | Roche | | |
| Trastuzumab | Genentech | anti-HER2 | ovarian cancer |
| HERCEPTIN | licensed to | | |
| | Hoffmann la | | |
| | Roche/ | | |
| | Immunogen | | |
| Palivizumab | Medimmune | | RSV |
| SYNAGIS | licensed to | | |
| | Abott | | |
| Alemtuzumab | BTG | anti-CD52 | leukemia |
| CAMPATH | licensed to | | |
| | Schering | | |
| ibritumomab | IDEC | anti-CD20 | NHL |
| tiuxetan | licensed to | | |
| ZEVALIN | Schering | | |
| Cetuximab | Merck/BMS/ | anti-HER1 | cancers |
| IMC-C225 | Imclone | | |
| Bevacizumab | Genentech/ | anti-VEGFR | cancers |
| AVASTIN | Hoffmann la | | |
| | Roche | | |
| Epratuzumab | Immumedics/ | anti-CD22 | cancers: non- |
| | Amgen | | Hodgkin |
| | | | lymphoma |
| Hu M195Mab | Protein Design | ND | cancers |
| | Labs | | |
| MDX-210 | Immuno-Designed | ND | cancers |
| | Molecules | | |
| BEC2 | Imclone | anti-GD3 | cancers |
| Mitumomab | | | |
| Oregovomab | Altarex | anti-CA125 | Ovarian cancer |
| OVAREX | | | |
| Ecromeximab | Kyowa-Hakko | anti-GD | malignant |
| KW-2971 | | | melanoma |
| ABX-EGF | Abgenix | EGF | cancers |
| MDX010 | Medarex | ND | cancers |
| XTL 002 | XTL | ND | anti-viral: HCV |
| | bio- | | |
| | pharmaceuticals | | |
| H11 SCFV | viventia | ND | cancers |
| | biotech | | |
| 4B5 | viventia | anti-GD2 | cancers |
| | biotech | | |
| XTL 001 | XTL | ND | anti-viral: HBV |
| | bio- | | |
| | pharmaceuticals | | |
| MDX-070 | MEDAREX | Anti-PSMA | Prostate cancer |
| TNX-901 | TANOX | anti-CD23 | |
| IDEC-114 | IDEC | inhibition | non-Hodgkin |
| | | ProteinC | lymphoma |

The invention also relates to the use of antibody described above, for producing a medicinal product intended to induce the expression of TNF, IFNγ, IP10, IL8 and Il-6 by natural effector cells of the immune system, said medicinal product being useful in particular for the treatment of cancer and of infections.

### FIGURE LEGENDS AND TITLES

FIG. 1: ADCC lysis of Raji cells, induced by anti-HLA-DR antibodies, expressed in CHO (triangle) or YB2/0 (square).

FIG. 2: Secretion of IL2 by Jurkat CD16 cells, induced by anti-HLA-DR antibodies, expressed in CHO (triangle) or YB2/0 (square).

FIG. 3: Correlation between the percentage of ADCC provided by NK cells and the secretion of IL2 by Jurkat CD16 cells.

FIG. 4: IL8 secreted by MNCs in the presence or absence of target.

FIG. 5: Secretion of cytokines by MNCs, induced by the anti-Rhesus antibodies (deduced value without target) Tox 324 03 062.

FIG. 6: Secretion of cytokines by polymorphonuclear cells, induced by the anti-Rhesus antibodies.

FIG. 7: Secretion of cytokines by NK cells, induced by the anti-Rhesus antibodies.

FIG. 8: Secretion of TNF alpha by NK cells, induced by the anti-CD20 and anti-HLA-DR antibodies expressed in CHO and YB2/0 (324 03 082).

FIG. 9: Secretion of IFN gamma by NK cells, induced by the anti-CD20 and anti-HLA-DR antibodies expressed in CHO and YB2/0 (324 03 082).

### EXAMPLE 1

### Anti-HLA-DR

1.1 CD16-Mediated ADCC Assay

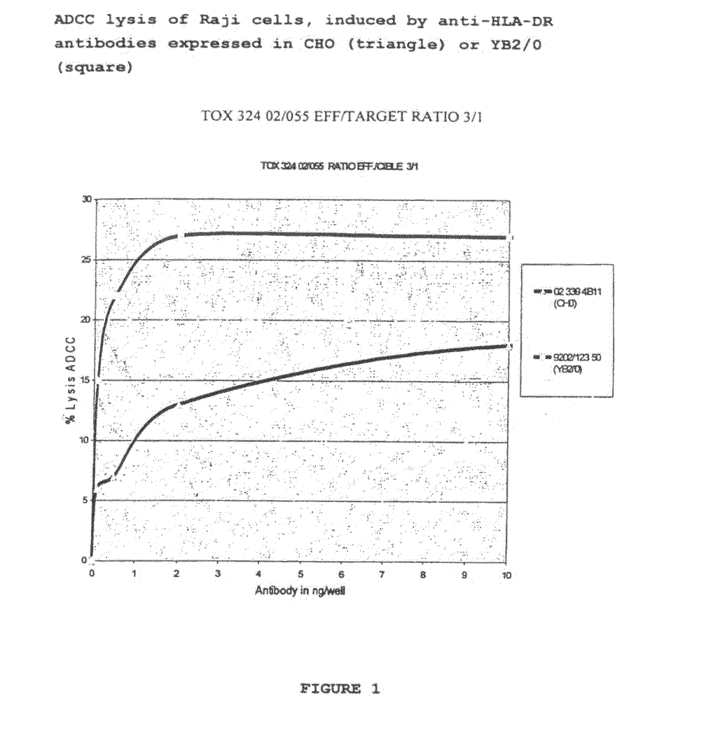

The chimeric anti-HLA-DR antibody was expressed in YB2/0 cells and in CHO cells. The chimeric anti-HLA-DR antibodies are capable of inducing cytotoxic activity against Raji cells expressing HLA-DR antigens at their surface. To do this, the same sequence encoding an IgG1 specific for the HLA-DR antigen is transfected into CHO and YB2/0. The antibodies are incubated with Raji cells (targets) and human natural killer (NK) cells. The cytotoxic activity of the antibodies on the Raji cells (ADCC) was evaluated after incubation for 16 h (see FIG. 1).

The two anti-HLA-DR antibodies expressed by YB2/0 (square) or CHO (triangle) induce Raji cell lysis by ADCC. The antibody expressed in YB2/0 is found to be more cytotoxic than CHO, especially under conditions of low amounts of effectors and low antibody concentrations.

1.2 IL-2 Assay

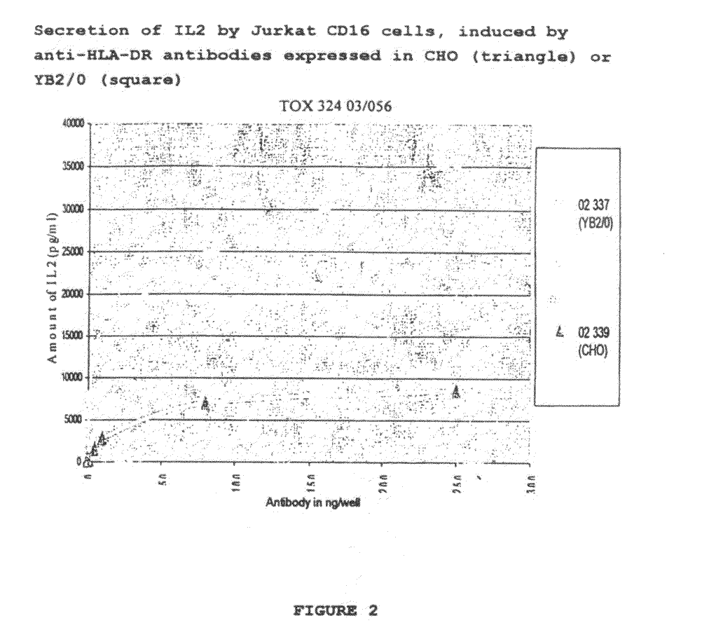

The same sequencing coding an IgG1 specific for the HLA-DR antigen is transfected into CHO and YB2/0. The antibodies are incubated with Raji cells (target) and Jurkat CD16 cells (effectors) carrying the amino acid phenylalanine (F) at position 158. The amount of cytokines (IL2) secreted by Jurkat CD16 is measured by ELISA (see FIG. 2).

The anti-HLA-DR antibodies induce a strong secretion of IL2 (cytokine). Comparatively, the secretion and therefore the degree of activation is greater when the antibody is expressed in YB2/0 (square) relative to CHO (triangle) at all the concentrations studied.

### EXAMPLE 2

### In Vitro Correlation Between ADCC and Release of IL-2 from Jurkat CD16

For this study, 3 anti-D monoclonal antibodies (Mabs) were compared.

The Mab DF5-EBV was produced by human B lymphocytes obtained from a D-negative immunized donor, and immortalized by transformation with EBV. This antibody was used as a negative control given that, in a clinical trial, it was shown to be incapable of eliminating rhesus-positive red blood cells from the circulation.

The monoclonal antibody (Mab) DF5-YB2/0 was obtained by expressing the primary sequence of DF5-EBV in the YB2/0 line. The monoclonal antibody R297 and other recombinant antibodies were also expressed in YB2/0.

These antibodies were assayed in vitro for their ability to induce lysis of papain-treated red blood cells using mononuclear cells (PBLs) as effector.

All the assays were carried out in the presence of human immunoglobulins (IVIgs) so as to reconstitute the physiological conditions.

It is thought that IVIgs bind with high affinity to FcgammaR1 (CD64). The two Mabs DF5-YB2/0 and R297 induce red blood cell lysis at a level comparable to that of the WinRho polyclonal antibodies. On the other hand, the Mab DF5-EBV is completely ineffective.

In a second series of experiments, purified NK cells and untreated red blood cells were used as effectors and targets, respectively. After incubation for 5 hours, the anti-D Mabs R297 and DF5-YB2/0 were shown to be capable of causing red blood cell lysis, whereas DF5-EBV remained ineffective.

In these two experiments, the red blood cell lysis was inhibited with the Mab 3G8 directed against FcgammaRIII (CD16).

In summary, these results demonstrate that the ADCC caused by Mab R297 and Mab DF5-YB2/0 involves FcgammaRIII expressed at the surface of NK cells.

In the context of the invention, a third series of experiments was carried out according to an in vitro assay using Jurkat CD16 cells in order to evaluate the effectiveness of anti-D antibodies. The Mabs were incubated overnight with Rhesus-positive red blood cells and Jurkat CD16 cells. The release of IL-2 into the supernatant was evaluated by ELISA.

A strong correlation between ADCC and Jurkat cell activation was observed, which implies that this assay can be used to discriminate between anti-D Mabs as a function of their reactivity toward FcgammaRIII (CD16).

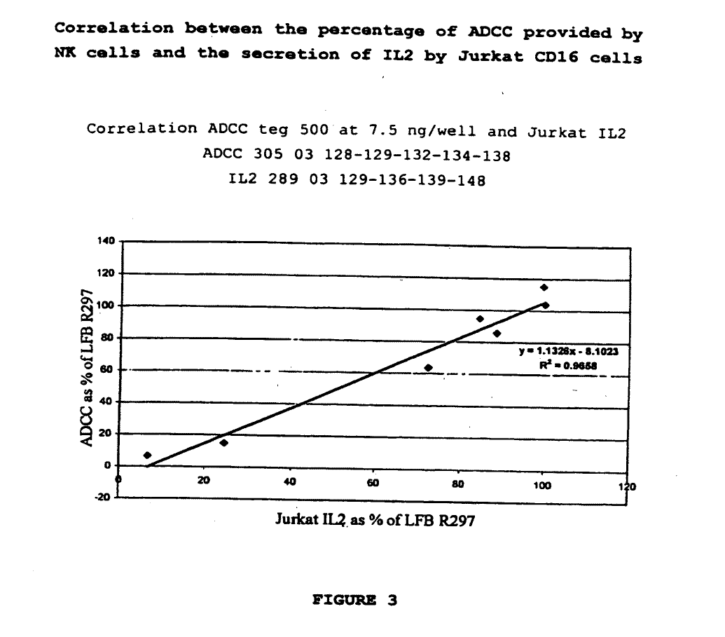

The same samples are evaluated by ADCC and in the Jurkat IL2 assay. The results are expressed as a percentage of the reference antibody “LFB-R297”. The curve for correlation between the 2 techniques has a coefficient r2 of 0.9658 (FIG. 3).

In conclusion, these data show the importance of post-translational modifications of the Fc portion of antibodies to their ability to induce FcgammaRIII-specific ADCC activity. The release of cytokines such as IL-2 correlates with the ADCC.

### EXAMPLE 3

### Activation of NK Cells and Production of IL2 and of IFNγ

Set-up model: Jurkat cell line transfected with the gene encoding the CD16 receptor. Applications: enhancement of an antitumor response. The IL2 produced by the activated effector cells induces activation of T lymphocytes and of NK cells that can go as far as stimulation of cell proliferation. The IFNγ stimulates the activity of CTLs and may enhance the activity of macrophages.

### EXAMPLE 4

### Activation of Monocyte-Macrophages and Production of TNF and IL-1Ra

Applications: enhancement of phagocytosis and induction of anti-inflammatory properties. The TNF produced by the activated effector cells stimulates the proliferation of tumor-infiltrating lymphocytes and macrophages. IL-1Ra is a cytokine which competes with IL1 at the level of its receptor and thus exerts an anti-inflammatory effect.

### EXAMPLE 5

### Activation of Dendritic Cells and Production of IL10

Applications: induction of tolerance specific to certain antigens. IL10 is a molecule that inhibits the activation of various effector cells, and the production of cytokines.

### EXAMPLE 6

### Induction of Cytokine Secretion by Various Effector Cells

Three cell populations were studied: polymorphonuclear cells, mononuclear cells and NK cells. Induction of cytokine synthesis is dependent on the presence of the target. There are few differences in the profiles of R297 and of the anti-Rhesus D polyclonal antibody, in terms of cytokines secreted by the effector cells. AD1 very commonly does not induce cytokine secretion.

Results:

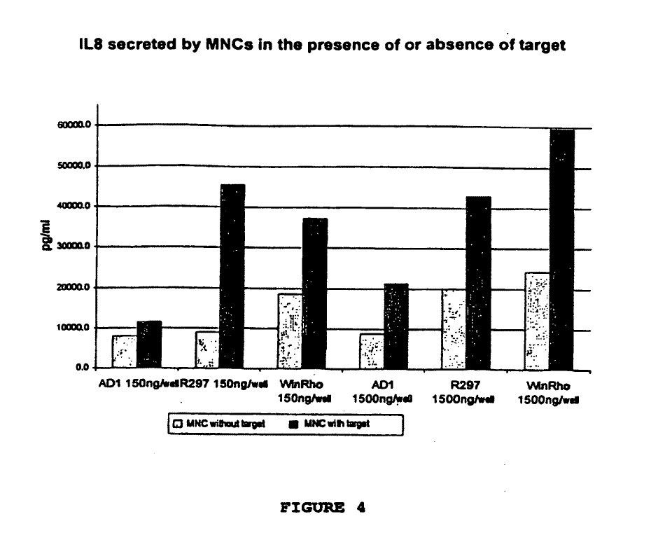

6.1 The anti-Rh D monoclonal antibody R297 and the anti-Rh D polyclonal antibody WinRho induce a considerable secretion of IL8 in the presence of mononuclear cells. This secretion is dependent on the antibody concentration and on the presence of the antigenic target. The antibody AD1 is much less effective at inducing the production of IL8 by mononuclear cells (FIG. 4).

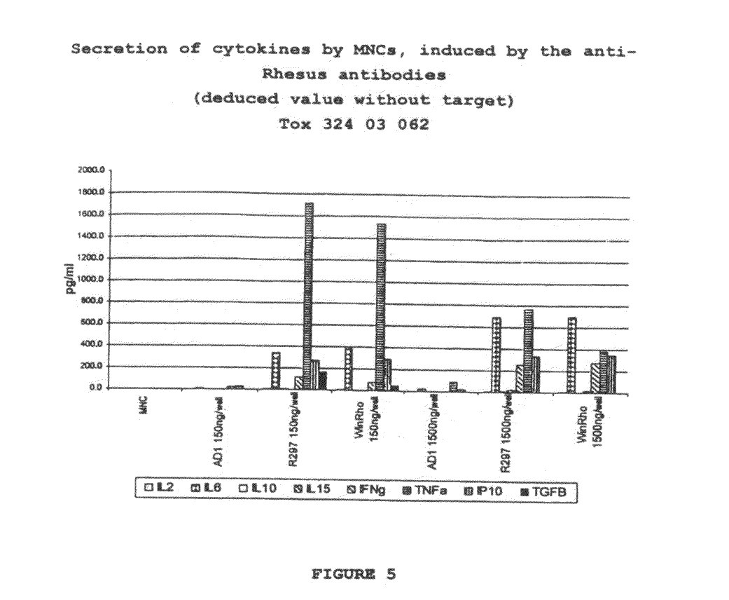

The anti-Rh D monoclonal antibody R297 and the anti-Rh D polyclonal antibody WinRho induce a considerable secretion of TNF alpha, and a less strong, although greater than that induced by AD1, secretion of IL6, IFN gamma, IP10, TNF alpha and TGF Beta, by mononuclear cells. This secretion increases as the antibody concentration increases for IL6, IFN gamma and IP10, but decreases for TNF alpha and TGF Beta (FIG. 5).

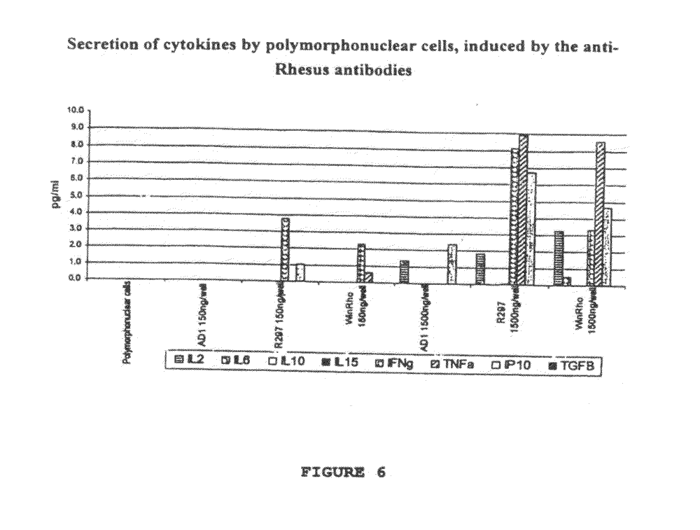

6.2 The anti-Rh D monoclonal antibody R297 and the anti-Rh D polyclonal antibody WinRho induce a very weak, but greater than AD1, secretion of IL2, IFN gamma, IP10 and TNF by polymorphonuclear cells. This secretion is antibody concentration-dependent (FIG. 6).

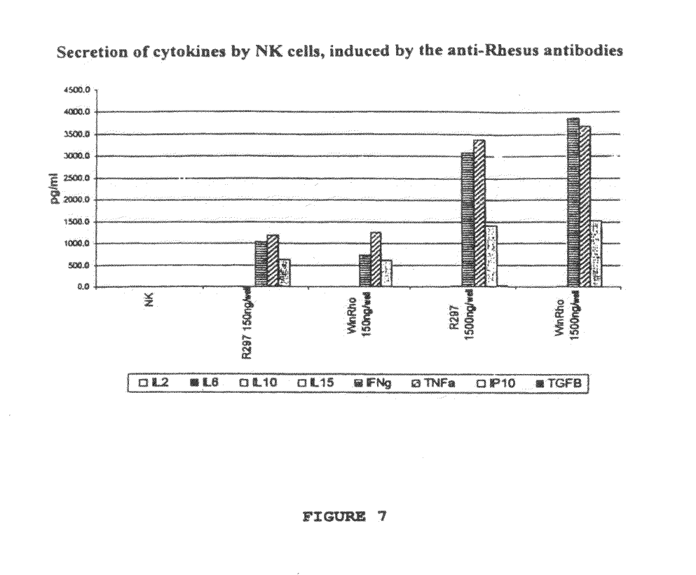

6.3 The monoclonal antibody R297 and the polyclonal antibody WinRho induce a considerable secretion of IFN gamma, IP10 and TNF by NK cells. This secretion is antibody concentration-dependent (FIG. 7).

### EXAMPLE 7

### Optimized Chimeric Anti-CD20 and Anti-HLA-DR Antibodies Produced in YB2/0

Introduction

Our first results showed that the anti-D antibodies produced in YB2/0 and also the polyclonal antibodies used clinically induced a strong ADCC activity and also the production of cytokines, in particular of TNF alpha and of interferon gamma (IFN gamma) from purified NK cells or from mononuclear cells. On the other hand, other anti-D antibodies, produced in other cell lines, are negative in ADCC and were found to be incapable of inducing this secretion of cytokines.

The additional results below show that this mechanism is not exclusive to the anti-D antibodies in the presence of Rhesus-positive red blood cells, but also applies to the anti-CD20 and anti-HLA-DR antibodies expressed in YB2/0. The expression in CHO confers on the antibody activating properties that are less substantial. This correlates with the results obtained in ADCC.

Materials

Antibodies

Anti-CD20: the chimeric anti-CD20 antibody transfected into YB2/0 is compared with a commercial anti-CD20 antibody produced in CHO (Rituxan).

Anti-HLA-DR: the same sequence encoding the chimeric anti-HLA-DR antibody is transfected into CHO (B11) or YB2/0 (4B7).

Target cells: Raji cells expressing at their surface the CD20 and HLA-DR antigen.

Effector cells: human NK cells purified by negative selection from human blood bags.

Method

Various concentrations of anti-CD20 or anti-HLA-DR antibodies are incubated with Raji cells and NK cells. After incubation for 16 hours, the cells are centrifuged. The supernatants are assayed for TNF alpha and IFN gamma.

Results:

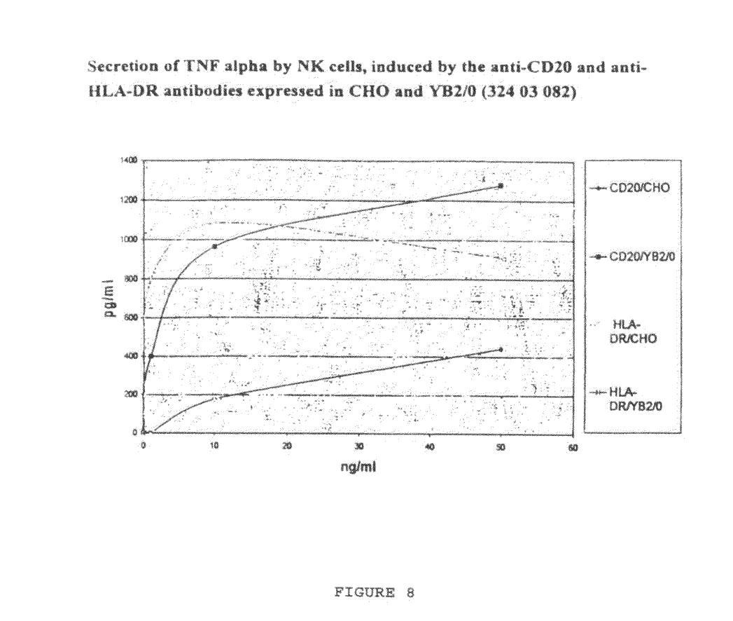

1) TNF alpha: the results are expressed in pg/ml of TNF alpha assayed in the supernatants. The various concentrations of antibodies added to the reaction mixture are given along the x-axis (FIG. 8).

The chimeric anti-CD20 and anti-HLA-DR antibodies produced in YB2/0 induce greater amounts of TNF in the presence of their target (Raji) compared with the same antibodies produced in CHO. The amount of TNF alpha is clearly dose-dependent on the concentration of antibody added. At 10 ng/ml of antibody, 5 times more TNF alpha is induced with the antibodies produced in YB2/0 compared with the antibodies produced in CHO.

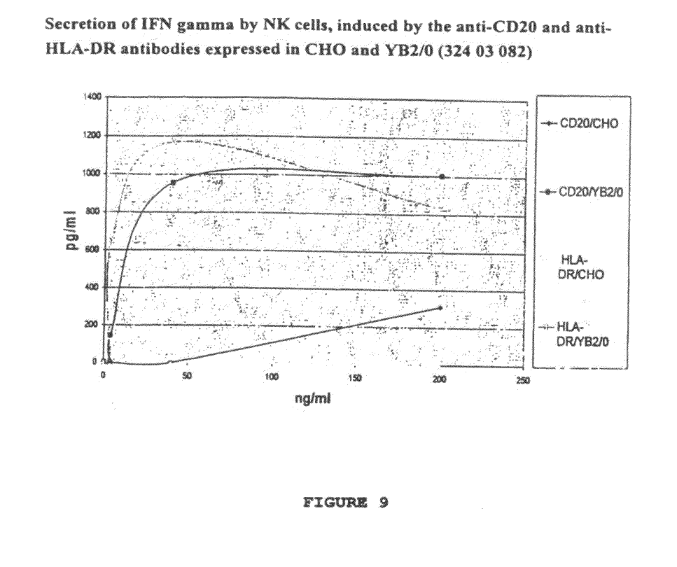

2) IFN gamma: the results are expressed in pg/ml of IFN gamma assayed in the supernatants. The various concentrations of antibodies added to the reaction mixture are given along the x-axis (FIG. 9).

The chimeric anti-CD20 and anti-HLA-DR antibodies produced in YB2/0 induce greater amounts of IFN gamma in the presence of their target (Raji) compared with the same antibodies produced in CHO. The amount of IFN gamma is clearly dose-dependent on the concentration of antibody added. At all the concentrations used (0 to 200 ng/ml), the anti-HLA-DR antibody produced in CHO induces no secretion of IFN gamma, whereas 40 ng/ml of the antibody produced in YB2/0 induces approximately 1000 pg/ml of IFN gamma.

For the anti-CD20 antibody, less than 10 ng/ml of the antibody produced in YB2/0, and 200 ng/ml of the antibody produced in CHO, are required in order to induce 300 pg/ml of IFN gamma (FIG. 9).

1. A human or humanized chimeric monoclonal antibody produced in a cell line selected for its properties of particular glycosylation of the Fc fragment of an antibody, or the glycan structure of which has been modified ex vivo, said antibody having an FcyRIII (CD16) -type ADCC rate of greater than 60%, 70%, 80% or preferably greater than 90%, compared with the same antibody produced in a CHO line or with a commercially available homologous antibody, characterized in that it has an ability to induce a rate of production of at least one cytokine by the Jurkat CD16 cell or a CD16 receptor-expressing effector cell of the immune system of greater than 60%, 100%, or preferably greater than 200%, compared with the same antibody produced in a CHO line or with a commercially available homologous antibody, and wherein said human, humanized, or chimeric monoclonal antibody is an anti-HLA-DR antibody.

2. The antibody as claimed in claim 1, wherein it has an ADCC rate of greater than 100% at a concentration of 10 ng/ml or less, compared with the same antibody produced in a CHO line or with a commercially available homologous antibody, and a rate of production of at least one cytokine by a CD16 receptor-expressing effector cell of the immune system of greater than 1000% at a concentration of 10 ng/ml or less, compared with the same antibody produced in a CHO line or with a commercially available homologous antibody.

3. The antibody as claimed in claim 1, wherein the cytokines that are released are interleukins.

4. The antibody as claimed in claim 1, wherein cytokines that are released are interferons.

5. The antibody as claimed in claim 1, wherein the cytokines that are released are tissue necrosis factors (TNFs).

6. The antibody as claimed in claim 1, wherein the antibody selected has the ability to induce the secretion of at least one cytokine chosen from IL-1, IL-2, IL-3, IL-4, IL-5, IL-6, IL-8, TNFa, TGFp, IP10 and IFNy, by the CD16 receptor-expressing effector cells.

7. The antibody as claimed in claim 1, wherein the antibody selected has the ability to induce the secretion of IL-2 by CD16 receptor-expressing effector cells of the immune system.

8. The antibody as claimed in claim 1, wherein the effector cell is a CD16 receptor-expressing Jurkat cell or by a leukocytic cell, in particular of the NK (natural killer) family, or by cells of the monocyte-macrophage group.

9. The antibody as claimed in claim 1, wherein it is produced in a cell line of the rat myeloma type, in particular YB2/0.

10. The antibody as claimed claim 1, wherein it is directed against an antigen of a pathological cell or of an organism that is pathogenic for humans, in particular against an antigen of a cancer cell.

11. The antibody as claimed in claim 10, wherein its specificity is anti-Rhesus D of human red blood cells.

12. The antibody as claimed in claim 11, wherein it is an anti-HLA-DR.

13. The antibody as claimed in claim 12, wherein it has an ADCC rate of greater than 100% at a concentration of 10 ng/ml or less, and a rate of IL-2 production by a CD16-receptor-expressing effector cell of the immune system of greater than up to 1000% at a concentration of 10 ng/ml or less, compared with the same antibody expressed in the CHO line, the expression line for Remitogen®.

14. The antibody as claimed in claim 12, wherein it is produced in a rat myeloma line, in particular YB2/0.

15. The antibody as claimed in claim 10, wherein it is an anti-CD20.

16. The antibody as claimed in claim 15, wherein it has an ADCC rate of greater than 100% at a concentration of 10 ng/ml or less, and a rate of IL-2 production by a CD16-receptor-expressing effector cell of the immune system of greater than up to 1000% at a concentration of 10 ng/ml or less, compared with Rituxan®.

17. The antibody as claimed in claim 15, wherein it is produced in a rat myeloma line, in particular YB2/0.

18. The antibody as claimed in claim 10, wherein it is selected from anti-Ep-CAM, anti-KIR3DL2, anti-VEGFR, anti-HER1, anti-HER2, anti-GD, anti-GD2, anti-GD3, anti-CD23, anti-CD30, anti-CD33, anti-CD38, anti-CD44, anti-CD52, anti-CA125 and anti-ProteinC; anti-Ep-CAM, anti-HER2, anti-CD52, anti-HER1, anti-GD3, anti-CA125 anti-GD, anti-GD2, anti-CD23 and antiProteinC; antivirals: HBV, HCV, HIV and RSV, anti-idiotypes specific for inhibitors, for example forclotting factors including FVIII and FIX.

19. The method of using an antibody as claimed in claim 1, for producing a medicinal product intended for the treatment of cancers and of infections with pathogenic agents.

20. The method of using an antibody as claimed in claim 1, for producing a medicinal product intended for the treatment of cancers of MHC class II-positive cells, in particular B-cell lymphomas, acute B-cell leukemias, Burkitt's lymphoma, Hodgkin's lymphoma, myeloid leukemias, T-cell lymphomas and leukemias, non-Hodgkin's lymphomas, and chronic 15 myeloid leukemias.

21. The method of using an antibody as claimed in claim 1, for producing a medicinal product intended to induce the expression of TNF, IP10 and IL-6 by natural effector cells of the immune system, said medicinal product being useful in particular for the treatment of cancer and of infections.
